(19) United States
(12) Patent Application Publication
(10) Pub. No.: US 2012/0304986 A1
(43) Pub. Date: Dec. 6, 2012

(54) THERAPEUTIC RESPIRATION DEVICE

(76) Inventors: Nikolai Grigorievich Lyapko, Donetskaya (UA); Aleksey Nikolaevich Lyapko, Donetsk (UA)

(21) Appl. No.: 13/577,154

(22) PCT Filed: Jan. 24, 2011
(86) PCT No.: PCT/UA2011/000002

§ 371 Date: Aug. 3, 2012

(30) Foreign Application Priority Data
Feb. 15, 2010 (UA) A 2010 01522

(51) Int. Cl. A61M 16/00; A61M 16/08

(52) U.S. Cl. 128/201.23

(57) ABSTRACT

Medical equipment for the prophylaxis and treatment of the respiration system with a reduced oxygen content in the air being breathed in, and can be used for training of the respiration and blood circulation system in an outpatient environment, and to determine the power-informational effect on a patient's body.

### TECHNICAL FIELD

The invention relates to a medical technology, particularly, to medical equipment for the prophylaxis and treatment of the respiration system with a reduced oxygen content in the air being breathed in, and can be used for training of the respiration and blood circulation system in outpatient environment, and to determine the power-informational effect on a patient's body.

### BACKGROUND ART

It is known from the prior art the therapeutic respiration device consisting of a container equipped with openings in the bottom and its upper section, a dosing device, and a respiration component (UA,26557,U).

The disadvantages of this known device are as follows: inconvenience in use, impossibility to regulate the concentration of hypoxic mixture in inhaled air, control of its formulation, and limited functions.

It is known so called “U SHYNSE” pyramid, the device for power (energy) influence upon living and nonliving natural beings which comprises a regular-shaped quadrangular carcass pyramid. Carcass elements forming lateral ribs of the pyramid contain at least one section that is made as a spiral, this spiral-type section being located in the middle part of the lateral carcass element (UA, 25 72069, C2).

The disadvantage of the above carcass device is the limitation on the scope of its functional capabilities, particularly the lack of using it for therapeutic respiration.

The most relevant device is a respirator for preparation of hypoxic-hypercapnic respiration mixture comprising a body being made of transparent material and having the opening for inflow and outflow of respiration mixture, and a seal unit having a fixing element for fixing the respirator on a patient's head. For obtaining a respiration mixture with various content of oxygen and carbon dioxide, the respirator is equipped with at least one barrier making of transparent material and having an opening, the opening for inflow and outflow of respiration mixture in the respirator's body being located in the place that is the most distant of a patient's face (SU, 1174043,A)

The disadvantages of the said device can be related also to the limitation of functional capabilities and insufficiency of a treating effect.

### SUMMARY OF INVENTION

The present invention aims at providing a device for therapeutic respiration in which by the improved body made as three-dimensional geometric figure, preferably, in the form of polyhedron, with at least one additional opening having controllable cross-section, and by the optimization of different forms of the ribs in such polyhedron, an extension of the scope of functional capabilities and the improved treating results are achieved.

This objective is achieved by providing a device for therapeutic respiration comprising a body having an opening for inflow and outflow of a respiration mixture. According to the invention the body is made as a three-dimensional geometric figure having ribs, preferably as polyhedron, having at least one additional opening with controllable cross section located in one of the side and/or at least on one rib of the body. Preferably at least one of the body side is made of elastic materials at least one side of the body being air-resistant.

In the preferred embodiments the side of the body may be is constructed to adjust the area of its permeable section or at least one side of the body may be made of at least two layers of material, at least one material layer on one side of the body being air-resistant or being made of metal.

In addition, an elastic container in the form of a bag could be fixed at least in one additional opening that is made in one side of the body.

It is also preferable to make a bottom of the body of elastic loose material having an controllable opening for fixing on a user's head or to construct the body as a pyramid having the dimension proportions of Cheops pyramid.

According to another embodiments of the claimed inventions it is proposed to make the ribs of the body as rods of dielectric and/or paramagnetic and/or ferromagnetic materials, at least one rib of the body having one opening, in which an electrically conducting rod is installed.

The electrically conducting rod installed in the body rib may have spirally wound insulated conductor or a spirally wound electrically insulated spiral-type conductor.

Furthermore at least one rib of the body could have a spirally wound electrically insulated conductor or a spirally wound electrically insulated spiral-type conductor.

Also it is preferably also that at least one group of conductor and/or spiral-type conductors wound on rods and located inside at least one rib of the body would be electrically connected with each other and fixed in at least one corner of the body or at least one group of conductor and/or spiral-type conductors spirally wound on ribs of the body would be electrically connected with each other and fixed in at least one corner of the body.

And finally, in another embodiment of the invention it is preferably to arrange the conductor ends wound on the body ribs and conductor ends wound on rods in its ribs and also electrically connect between each other in at least one corner of the body.

It is also preferably that one corner of the body contained a fixed electrically conducting rod in the form of antenna.

### BRIEF DESCRIPTION OF DRAWINGS

The invention is described with reference to, though not limited by, the following drawings, in which:

FIG. 1 is a general view of the device in accordance with the invention having the body in the form of pyramid;

FIG. 2—the device in accordance with the invention having the body in the form of polyhedron;

FIG. 3—the device in accordance with the invention comprising an elastic loose bottom;

FIG. 4—the device in accordance with the invention having the elastic container (bag) fixed in additional opening at one rib of the body;

FIG. 5—the device in accordance with the invention having air-resistant rib of the body which is constructed to adjust the area of its permeable section using zipper locks installed at the ribs of the body;

FIG. 6 is a cross section along the line A-A line in FIG. 3 on an enlarged scale representing the rod installed inside the rib, with the joint conductor and spiral-type conductor spirally wound around it;

FIG. 7 is a cross section along the line A-A in FIG. 3 on an enlarged scale representing the rod installed inside the rib, with the separate conductor and spiral-type conductor spirally wound around it;

FIG. 8 is the part of the body rib on an enlarged view which demonstrates the joint conductor and spiral-type conductor spirally wound around it;

FIG. 9 is the part of the body rib on an enlarged scale which demonstrates the separate conductor and spiral-type conductor spirally wound around it;

FIG. 10 is a general view of the device in accordance with the invention having an electrically conducting antenna-type rod;

FIG. 11 is a fragment, on an enlarged scale, of the connection of ribs and separately wound at each rib the conductor and spiral-type conductor at one corner of the body;

FIG. 12—is a fragment, on an enlarged scale, of the connection of the ribs and jointly wound at each rib the conductor and spiral-type conductor at one corner of the body;

FIG. 13 is a fragment, on an enlarged scale, of the connection of ribs and spiral-type conductor wound at each rib at one corner of the body.

### BEST MODE FOR CARRYING OUT OF THE INVENTION

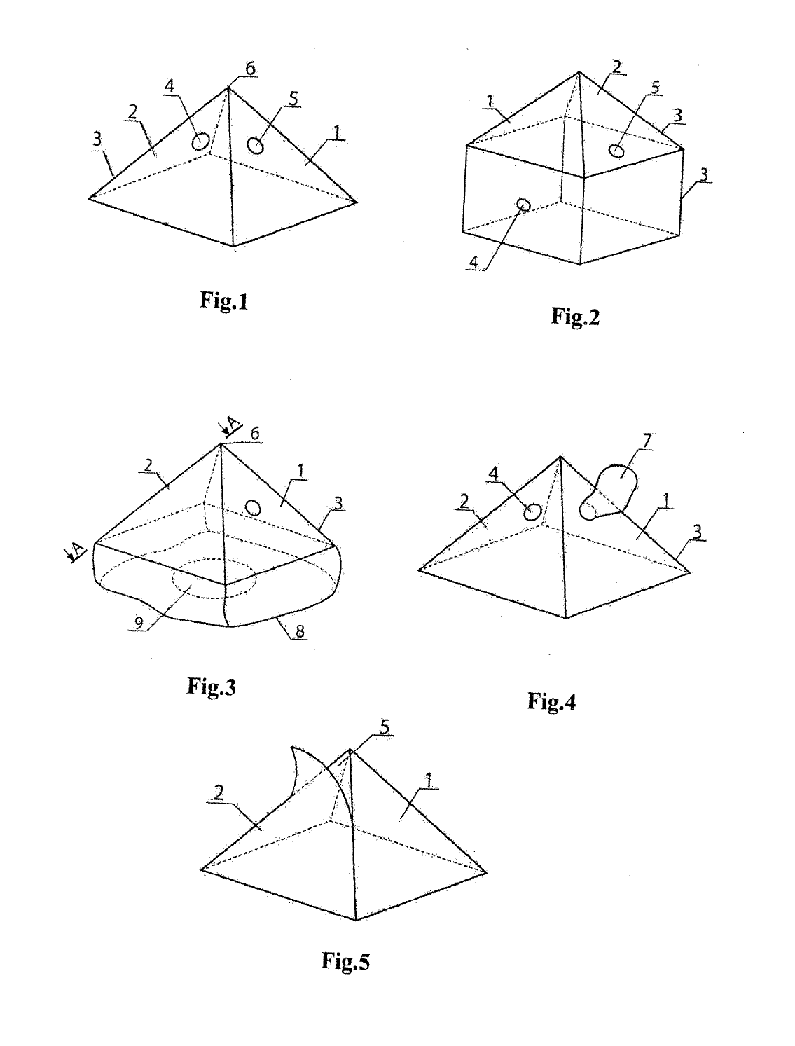

As shown in FIG. 1-5, the claimed device for therapeutic respiration comprises the body 1 which is made as a three-dimensional geometric figure having ribs, preferably as polyhedron (FIG. 2), in particular as pyramid (FIG. 1, 3, 4, 5). Body 1 has side 2 including one or several layers of material in the preferred embodiments, side 2 being made of elastic material or air-resistant or with adjusted area of its permeable section. One layer of side 2 is air-resistant and/or metallic. In accordance with different preferred embodiments of the invention, ribs 3 of body 1 are made as rods of dielectric and/or paramagnetic and/or ferromagnetic materials which are joined at the corner of the body 1.

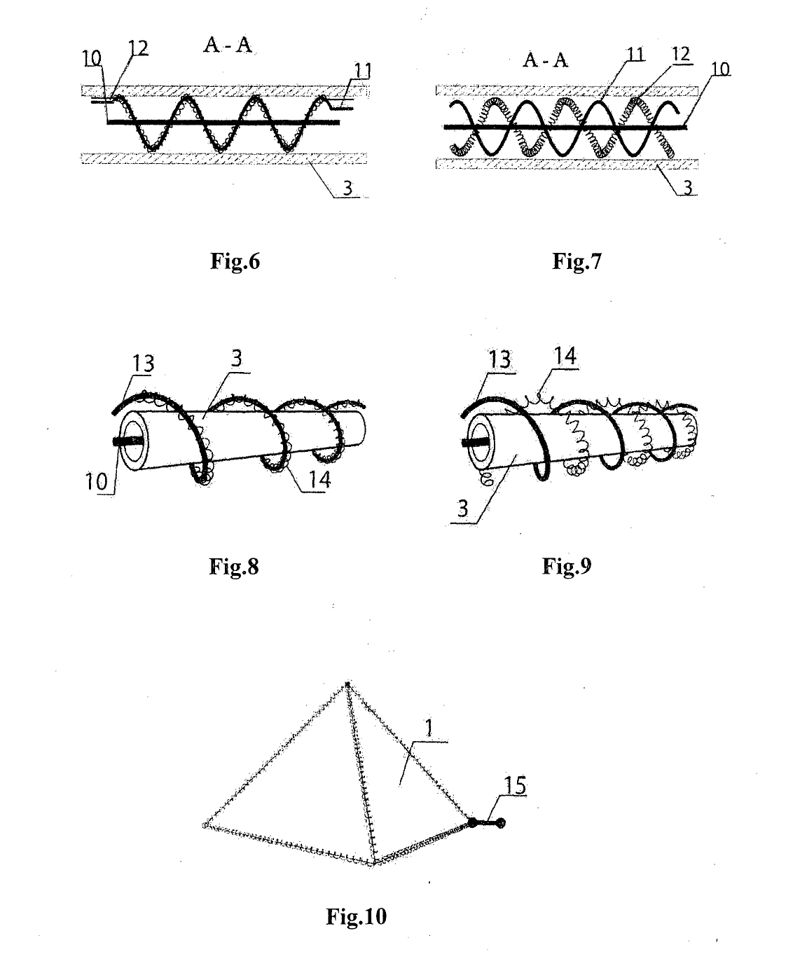

The body 1 has an opening 4 for inflow and outflow of a respiration mixture and at least one additional opening 5 with controllable cross section located in one of the side 2 and/or at least on one rib 3. In one of the preferred embodiment the additional opening 5 is formed by using zipper which is fixed, for example, on the ribs 3 as it is shown on FIG. 5. The ribs 3 are joined at the corner 6 of the body 1. At another embodiment of the claimed invention shown on FIG. 4 an elastic container 7 in form of a bag is fixed within the additional opening 5 of the body 1. According to the other embodiment of the invention that is shown on FIG. 3 the bottom 8 of the body 1 is made of elastic loose material having an controllable opening 9 for passing through a user's head and fixing on a user's neck or body. An electrically conducting rod 10 is preferably installed into one of a hollow ribs 3 (FIG. 6-7). In addition an electrically insulated conductor 11 and/or electrically insulated spiral-type conductor 12 may be spirally wound on the rob 10. An electrically insulated conductor 13 and/or electrically insulated spiral-type conductor 14 may be also spirally wound on at least one rib 3 of the body 1 (FIG. 8-9). In the other preferred embodiment the claimed invention has the electrically conducting rod 15 that is fixed in the form of antenna at least at one the corner 6 of the connection of ribs 3 of the body 1 (FIG. 10).

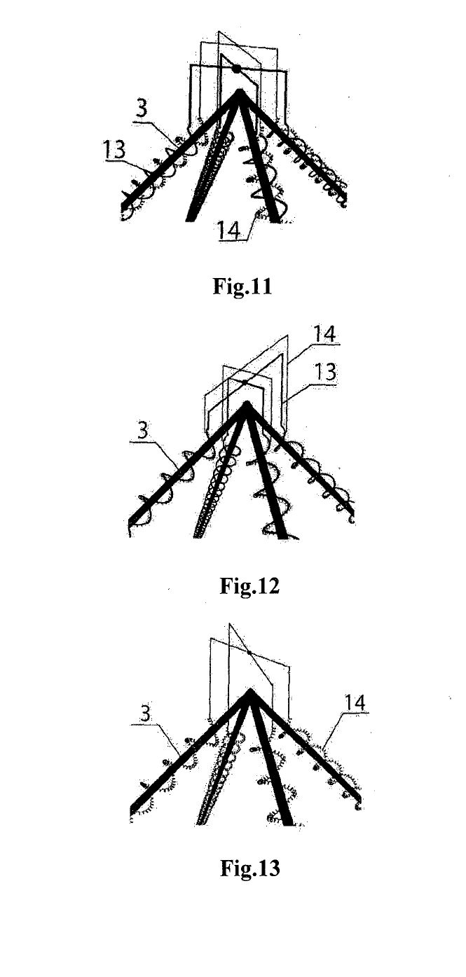

Furthermore, for increasing the power and information influence on patient's organism without disturbance of the inherent electromagnetic field it is proposed to connect electrically conducting rods 10 (rids 3) at the corners of the body 1 and/or outputs of the group of conductors or all conductors 11,12,13,14 as shown on FIG. 11-13. In particular FIG. 11 demonstrates a fragment of connection at one corner of the body 1 the ribs 3 and separately wound conductors 13 and 14, FIG. 12—ribs 3 and conductor 13 that is jointly wound with a spiral-type conductor 14, FIG. 13—ribs 13 and separately wound spiral-type conductor 14. It is clear that within the scope of the claimed invention there may be another embodiments of connection of conductors 11,12,13,14 at the corner of connection of ribs 3 or rod 10 of the body 1.

The device operates as follows.

The device is usually fixed on a patient's head through an controllable opening 9 that is made in bottom 8 of body 1. In the initial period, opening 4 and/or opening 5 are located in an open position, with the largest area of cross section. Body 1 is filled with air that is inhaled by the patient and that partially outflows through an open opening 4 and/or 5. This air has high content of carbon dioxide. When inhaling, inside of the space of body 1 it is formed bioactive respiration mixture containing the air inhaled by a patient and atmospheric air (incoming through open opening 4 and/or 5) and having the increased content of CO₂ and slightly lowered content of O₂.

At the initial stage, when a patient's head is located in the device, the respiration mixture contains about 0.3% carbon dioxide (such mixture is inhaled by spacemen during flights on space ships). At the final stage, the respiration mixture contains 2-2.5% of carbon dioxide, which is 60-80 times above its content in the atmospheric air. Content of carbon dioxide is controlled by changing of cross section along at least one additional opening 5, which made in one of side 2 and/or rib 3—in the form of zipper lock. Due to use of air-resistant materials for side with one or two layers, availability to change the flow cross section of such side it is possible to control supply of atmospheric air to body 1 and outflow of carbon dioxide out of it. At the final stage, the openings are maximally overlapped resulting in reduction of the amount of atmospheric air incoming to body 1. Such system of stage-by-stage transition ensures adjustment of content of breathing mixture in compliance with individual needs of a patient to the composition of inhaled mixture and ensures efficient treatment of various diseases. Elastic container 7 additionally accumulates respiration mixture and enhances mixing of atmospheric air inhaled by a patient.

Due to the fact that the construction of body 1 is made as a three-dimensional geometric figure having ribs 3, preferably as polyhedron, and due to the different embodiments of these ribs namely the ribs 3 may be made in the form of rods of dielectric and/or paramagnetic and/or ferromagnetic materials or may be made hollow with installed electrically conducting rods in appropriate openings with spirally wound electrically insulated conductors 11 and/or conductors 12 on rods 10, and with specially wound electrically insulated conductors 13 and/or spiral-type conductors 14, and due to the installation of electrically conducting antenna 15 ensures additional power-informational effect to a patient's body during respiration, which in its turn improves cumulative treating effect.

To the author's opinion, the claimed device, besides providing the controllable limitation of gas exchange of air mixture inside the body (pyramid) with air mixture outside the pyramid and environmental air, is able to additionally selectively adjust and transform frequency components of environmental air, additionally releasing that frequency range, which will cover the frequency component of ribs of geometric space figure, e.g., cube, polyhedron, pyramid etc. Thus, for example, the rib of polyhedron is able to transform all range of frequencies of environmental air, if the rib has been inserted with additional metal rod or tube, where wave frequency features of such rod or tube will resonate with environmental poly-frequency wave components of environmental air, resonating higher-frequency part of frequency range. If a spiral-type metal or non-metal element is installed on such rod, tube or instead of them, its wave frequency properties will preferably release and resonate with medium high-frequency range of waves of environment. If any rib (ribs) of polyhedron is additionally equipped with small spiral-type component that is wound in a larger spiral together with the above-mentioned components or instead of them, the resonating frequency and wave properties of such component will cover low-frequency or medium-frequency part of waves of environmental air. In that case selected wave types are formed on the ribs and inside the polyhedron (polyhedrons) with selected frequencies and with wide range of higher-density frequencies in air space inside the polyhedron, which is fixed on a user's head and body.

The proposed device for therapeutic respiration has a simple construction, operates reliably, is portably and safely, requires no artificial gas component sources, may be used for prevention and treatment purposes in the outpatient setting and at home and ensures increase of non-specific resistance of both sick and healthy persons, particularly, to reduce the side effects during drug therapy (e.g., chemotherapy).

1. A device for therapeutic respiration comprising a body having an opening for inflow and outflow of a respiration mixture, characterised in that the body is made as a three-dimensional geometric figure having ribs, preferably as polyhedron, having at least one additional opening with controllable cross section located in one of the side and/or at least on one rib of the body.

2. The device according to claim 1, characterised in that at least one of the body side is made of elastic materials.

3. The device according to claim 1, characterised in that at least one side of the body is air-resistant.

4. The device according to claim 1, characterised in the side of the body is constructed to adjust the area of its permeable section.

5. The device according to claim 1, characterised in that at least one side of the body is made of at least two layers of material.

6. The device according to claim 1, characterised in that at least one material layer on one side of the body is air-resistant.

7. The device according to claim 1, characterised in that at least one material layer of the body side is made of metal.

8. The device according to claim 1, characterised in that an elastic container in the form of a bag is fixed at least in one additional opening that is made in one side of the body.

9. The device according to claim 1, characterised in that a bottom of the body is made of elastic loose material having an controllable opening for fixing on a user's head.

10. The device according to claim 1, characterised in that the body is constructed as a pyramid having the dimension proportions of Cheops pyramid.

11. The device according to claim 1, characterised in that the ribs of the body are made as rods of dielectric and/or paramagnetic and/or ferromagnetic materials.

12. The device of claim 1, wherein least at one rib of the body has one opening, in which an electrically conducting rod is installed.

13. The device of claim 12, wherein the electrically conducting rod installed in the body rib has a spirally wound insulated conductor.

14. The device of claim 12, wherein the electrically conducting rod installed in the body rib has a spirally wound electrically insulated spiral-type conductor.

15. The device of claim 1, wherein at least one rib of the body has a spirally wound electrically insulated conductor.

16. The device of claim 1, wherein at least one rib of the body has a spirally wound electrically insulated spiral-type conductor.

17. The device of claim 1, wherein at least one group of conductor and/or spiral-type conductors wound on rods and located inside at least one rib of the body is electrically connected with each other and fixed in at least one corner of the body.

18. The device of claim 1, wherein at least one group of conductor and/or spiral-type conductors spirally wound on ribs of the body is electrically connected with each other and fixed in at least one corner of the body.

19. The device of claim 1, wherein outputs of conductors and/or spiral-type conductors wound on ribs of the body, and outputs of conductors and/or spiral-type conductors wound on rods installed in its ribs are electrically connected with each other and fixed in at least one corner of the body.

20. The device of claim 1, wherein at least in a corner of the body, an electrically conducting rod is fixed in the form of an antenna.
